(19) United States
(12) Plant Patent Application Publication
(10) Pub. No.: US 2002/0188985 P1
(43) Pub. Date: Dec. 12, 2002

(54) Chrysanthemum plant named 'MOZA RT 01'

(76) Inventor: Frank Moser, Alva, FL (US)

Correspondence Address: KNOBBE MARTENS OLSON & BEAR LLP, 2040 MAIN STREET, FOURTEENTH FLOOR, IRVINE, CA, 92614, US

(21) Appl. No.: 09/865,147

(22) Filed: May 24, 2001

(51) Int. Cl. A01H 5/00

(52) U.S. Cl. PLT/298

(57) ABSTRACT

A new and distinct Chrysanthemum plant cultivar characterized by a daisy type inflorescence, a consistent flowering response to short days, blooming consistently after 45 days of short day length, a free branching habit, a consistent natural season flowering habit during the first two weeks of September, and dark purple/red ray florets.

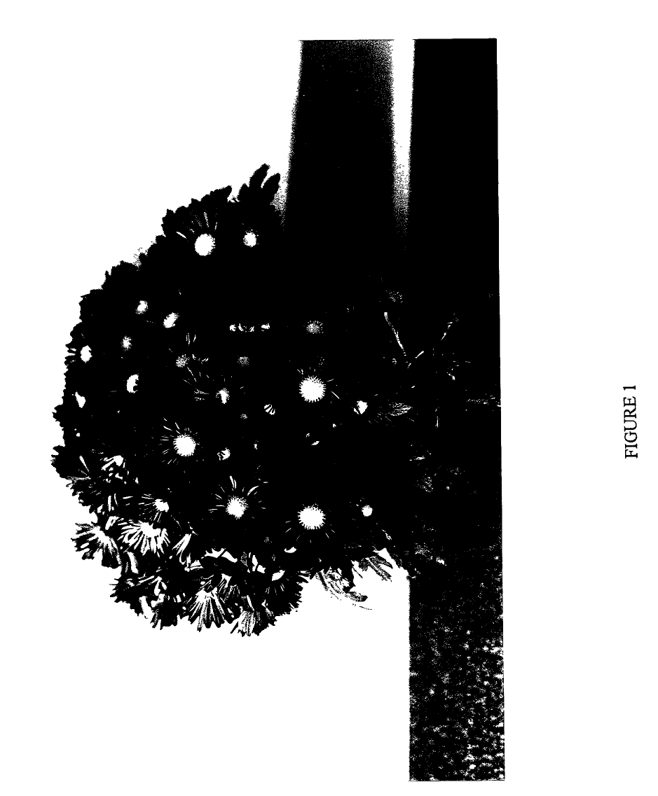

### BACKGROUND OF THE INVENTION

The present invention relates to a new and distinct cultivar of Chrysanthemum plant, botanically known as Dendranthema morifolium and hereinafter referred to by the cultivar name ‘MOZA RT 01.’

The new cultivar is a product of induced mutation by radiation. The new cultivar originated by exposing an entire plant of the Chrysanthemum cultivar ‘MZT TMP8’ (U.S. patent application Ser. No. 09/243,748) to radiation. This cultivar was discovered and selected by the inventor in November 1998.

Asexual reproduction of the new cultivar by apical tip cutting and meristem tissue culture was performed in Oxnard, Calif. and has shown that the unique features of this cultivar are stable and reproduced true to type on successive generations.

### SUMMARY OF THE IVENTION

The cultivar ‘MOZA RT 01’ has not been observed under all possible environmental conditions. The phenotype may vary somewhat with variations in environment such as temperature, daylength, and light intensity, without, however, any variance in genotype.

The following traits have been repeatedly observed and are determined to be the unique characteristics of ‘MOZA RT 01.’ These characteristics in combination distinguish ‘MOZA RT 01’ as a new and distinct Chrysanthemum cultivar:

1. Daisy type inflorescence;

2. Consistent flowering response to short days, blooming consistently after 45 days of short day length;

3. Free branching habit;

4. Consistent natural season flowering habit during the first two weeks of September; and

5. Dark purple/red ray florets.

Plants of the new cultivar are similar to plants of the parent cultivar, ‘MZT TMP8’ in most horticultural characteristics, however plants of the new cultivar differed from plants of the cultivar ‘MZT TMP8’ primarily in ray floret color, number of ray florets, and number of disc florets. The new cultivar ‘MOZA RT 01’ has darker, more reddish purple ray florets than the parent cultivar. The new cultivar has fewer ray florets than the parent cultivar, with an average of 80 florets compared to the parent's 110 florets, in similar cultural conditions. The new cultivar also has more disc florets than the parent.

### BRIEF DESCRIPTION OF THE BOTANICAL

The photograph shows a plant of the new cultivar which was grown in a 2-gallon container. A single cutting was grown in the container, and the plant was grown in outdoor field conditions.

### DETAILED BOTANICAL DESCRIPTION

In the following description, color references are made to The Royal Horticultural Society Colour Chart except where general terms of ordinary dictionary significance are used. The following observations and measurements describe plants grown in a commercial field in Oxnard, Calif. during the month of September 2000. The growing temperature ranged from 60° to 65° F. at night to 70° to 85° F. during the day. Measurements and numerical values represent averages of typical flowering types.

Botanical classification: Dendranthema morifolium cultivar ‘MOZA RT 01’

Commercial classification: Garden-type Chrysanthemum

Parentage: Induced mutation of Dendranthema morifolium cultivar ‘MZT TMP8’

Propagation

Time to rooting.—7 to 14 days at approximately 21° C.

Rooting habit.—Fine, fibrous

Plant

Growth habit.—mounding herbaceous perennial

Height.—Approximately 25 cm

Growth rate.—Moderate

Branching characteristics.—Free Branching

Foliage

Leaf.—Arrangement: Alternate single Average Length: Approximately 6.5 cm Average Width: Approximately 3.5 cm Shape of blade: Ovate Apex: Acuminate Base: Attenuate Attachment: Stalked Margin: Serrate Texture of top surface: lightly pubescent Texture of bottom surface: lightly pubescent Color: youngfoliage upper side: Near 137A young foliage under side: Near 137B mature foliage upper side: Near 137A mature foliage under side: Near 137B venation upper side: Near 137C venation under side: Near 137D

Petiole.—Average Length: Approximately 2.0 cm

Bloom

Inflorescence.—Flowering habit: induced by darkness period greater than 13.5 hours, approximately 45 days of appropriate day length required to induce and develop blooms Inflorescenceform: Daisy Naturalflowering season: Approximately the first two weeks of September Number of inflorescences per stem: Approximately 9 Inflorescence diameter: Approximately 4.5 cm Inflorescence depth: 1.5 cm Disc diameter: 1.5 cm Inflorescence longevity on plant: Approximately 18-22 days Persistence: Persistent

Ray florets.—Appearance: Matte Texture: Smooth Average Number per flower: Approximately 80 Shape: Oblanceolate Margin: Entire Apex: Obtuse Length: Approximately 2.0 cm Width: Approximately 0.5 cm Color: upper surface at first opening: Near 59A upper surface at maturity: Near 59D upper surface at fading: Near 60A under surface at first opening: Near 79A under surface at maturity: Near 70B under surface at fading: Near 70B

Disc florets.—Appearance: Shiny Texture: Smooth Average number per flower: Approximately 200 Shape: Cylindric Apex: Obtuse Average Length: Approximately 0.7 cm Average Width: Approximately 0.1 cm Color: at first opening: Near 46A at maturity: Near 16A at fading: Near 22A

Peduncle.—Length: at terminal end (shortest): Approximately 4 cm at lateral end (longest): Approximately 6 cm Strength: Moderate Color: Near 137C Habit: Erect

Inflorescence bud.—Length: Approximately 0.8 cm Diameter: Approximately 0.8 cm Form: Globular Color: Near 138A

Sepals.—Appearance: Matte Texture: Lightly pubescent Number: Approximately 30 to 40 Shape: Ovate Margin: Entire Apex: Acute Color: upper side: Near 137B under side: Near 137B

Reproductive Organs

Ray florets.—Pistillate: Number ofpistils per flower: 1 Stigma shape: 2 branched Stigma color: Near 7D Stigma length: 0.6 cm Style color: Near 7D

Disc Florets.—Pistillate: Number ofpistils per flower: 1 Stigma shape: Cylindric Style length: 0.5 cm Staminate: Number of stamens per flower: Approximately 5 Anther shape: Tubular Anther color: Near 14C Pollen color: Near 15A

‘MOZA RT 01’: Table of R.H.S. Color Codes

| | | | |
|---|---|---|---|
| FOLIAGE | Leaf | young foliage upper surface | Near 137A |
| | | young foliage under surface | Near 137B |
| | | mature foliage upper surface | Near 137A |
| | | mature foliage under surface | Near 137B |
| | | venation upper surface | Near 137C |
| | | venation under surface | Near 137D |
| BLOOM | Ray Florets | upper surface at first opening | Near 59A |
| | | upper surface at maturity | Near 59D |
| | | upper surface at fading | Near 60A |
| | | under surface at first opening | Near 79A |
| | | under surface at maturity | Near 70B |
| | | under surface at fading | Near 70B |
| | | Pistillate: Stigma | Near 7D |
| | | Pistillate: Style | Near 7D |
| | Disc Florets | at first opening | Near 46A |
| | | at maturity | Near 16A |
| | | at fading | Near 22A |
| | | Staminate: Anther | Near 14C |
| | | Staminate: Pollen | Near 15A |
| | Peduncle | | Near 137C |
| | Inflorescence | | Near 138A |
| | Bud | | |
| | Sepals | upper surface | Near 137B |
| | | under surface | Near 137B |

What is claimed is:

1. A new and distinct cultivar of Chrysanthemum plant named ‘MOZA RT 01’ as herein illustrated and described.
